(19) United States
(12) Patent Application Publication
(10) Pub. No.: US 2018/0078180 A1
(43) Pub. Date: Mar. 22, 2018

(54) OCCUPANCY MONITORING DEVICE

(71) Applicant: SAFEKEEPING SOLUTIONS LTD, Worcester Worcestershire (GB)

(72) Inventors: Ruth ALLEN, Worcester (GB); Andrew ASHCROFT, Totton Southampton (GB)

(73) Assignee: SAFEKEEPING SOLUTIONS LTD, Worcester Worcestershire (GB)

(21) Appl. No.: 15/563,792

(22) PCT Filed: Mar. 31, 2016
(86) PCT No.: PCT/GB2016/000068

§ 371 Date: Oct. 2, 2017

(30) Foreign Application Priority Data
Apr. 1, 2015 (GB) 1505627.8
Mar. 30, 2016 (GB) 1605282.1

(51) Int. Cl. A61B 5/11; A61B 5/00; G08B 21/22; H03K 17/955

(52) U.S. Cl. CPC A61B 5/1115; A61B 5/6892; A61B 5/1117; H03K 2217/960715; G08B 21/22; H03K 17/955; H03K 2217/96078; A61B 5/746

(57) ABSTRACT

An occupancy monitoring device, such as for a bed or chair, including a capacitor having a first electrode adapted for attachment along the side of a bed or mattress or a chair, a second electrode adapted for positioning remotely from the first electrode and a defector tor detecting dielectric shift induced in the capacitor by movement of an occupant on the bed, mattress or chair.

This invention relates to an occupancy monitoring device, in particular to a method and system for the detection of a person attempting to dismount a bed or chair.

### TECHNICAL FIELD

The high incidence of inpatient falls is a well-documented major clinical governance issue and a substantial healthcare burden. Across England and Wales around 280,000 patients fall in NHS inpatient settings per year, as reported by the National Reporting and Learning System Survey 2010. Elderly patients are particularly vulnerable; The National Patient Safety Agency reported in 2010 that 82% of falls occur in patient over 65 years old and 67% in patients aged over 75 years. At least a quarter of all falls in hospital are falls from a bed (National Patient Safety Agency 2007). Analysis of incident reports shows that most falls are not witnessed by staff (National Patient Safety Agency 2007).

Many falls from a bed or chair could be avoided if the vulnerable person is given assistance while he or she dismounts the bed or chair but the person is often unwilling or unable to summon assistance. In other cases the occupant may be confused or disorientated so that he or she is not aware that such assistance is necessary.

Equipment does already exist to detect persons leaving a bed or a chair. Most commercially-available devices use some form of pressure-activated switch as their sensitive element. These are often in the form of a pad placed centrally within the mattress or seat of the chair, which detects the release of pressure as the occupant moves his or her weight from the mattress/seat. In other cases pressure pads are placed on the floor to detect the footfall as the occupant leaves the bed/chair. While simple and economical, this technology only triggers an alarm after the occupant has substantially vacated the bed or chair by which time the vulnerable person may already have been exposed to significant risk of injury.

For simplicity, pressure sensors are most often switches that are triggered when pressure exceeds or falls below a certain threshold value. Setting this threshold is difficult for a broad range of scenarios (e.g. patient weight) and settings are prone to error. Moreover, the use of a threshold sensor results in a binary signal (i.e. the device is either triggered or it is not) and this limits the scope for detection algorithms and false alarm rejection techniques. Users often report lack of confidence in these devices due to the very high rate of false alarms. Furthermore, pressure pads are frequently bulky, which can cause discomfort to the occupant or, in some embodiments, may limit freedom of movement to an unacceptable extent.

Previous inventions have solved the obvious limitations of pressure-sensitive devices by replacing the pressure actuated switch with a capacitive sensor, such as that described in WO 98/10391 (Newham, Paul). These sensors typically operate by placing the occupant of the bed within the electric field between the electrodes of a capacitance array that is housed within a polymer mat and then sensing the changes in the capacitance brought about by the dielectric shift brought about by the proximity or absence thereof of the patient's body mass. Changes in capacitance are most often detected by the change in frequency of oscillation of a tuned L-C (inductor-capacitor) oscillator circuit wherein the sensor acts as a variable capacitor.

These capacitive arrays can be realised as thin membranes that present no discomfort to the occupant of the bed but these types of sensor have a defined geometry and placement. Generally, the sensor is placed near the larger mass areas of the patient, preferably being positioned beneath the upper torso portion of the patient. However, movement of this part of the body is not necessary indicative of a patient's intention to leave the bed, for example, a patient may simply be sitting up to eat or leaning forward to take a drink. Furthermore, the sensor suffers from further drawbacks. For example, the positioning of the mat across the bed can be affected by profiling of the bed and the positioning of pillows between the patient's torso and mattress may interfere with the signaling of the device.

It is an aim of the present invention to provide an improved occupancy monitoring device, in particular a device for monitoring the occupancy of a bed or chair that overcomes, or at least alleviates, the abovementioned drawbacks.

A further aim of the present invention is to provide a method of detecting an intention of a person to dismount from a bed or chair that overcomes, or at least alleviates the above mentioned drawbacks in the art.

### SUMMARY OF THE INVENTION

Accordingly, a first aspect of the present invention provides an occupancy monitoring device for detecting movement of a load on an object, the device comprising a sensor in the form of a capacitor having a first electrode adapted for attachment to at least part of the object and a second electrode adapted for positioning remotely from the first electrode, the device further comprising a detector for detecting dielectric shift induced in the capacitor by movement of a load on the object.

The first and second electrodes provide a continuous stream of capacitance data enabling detailed information to be inferred about the position of the load on the object.

Preferably, the load is a person and the object is preferably a bed, mattress, or covering for the same. Alternatively, the object may comprise a chair.

The first electrode may comprise a single conductor in the form of, for example, an elongated wire, film, tape or fibre for attachment to the object. It is preferable for the first electrode to be attached at or close to a lateral edge of the bed or mattress, preferably parallel to a longitudinal axis of the object. A recumbent person having an intention to leave the bed will generally, as a first action, extend a limb towards the edge of the bed/mattress. Placement of the electrode along the side of the bed means that the limb will have a high probability of intersecting the electrode thus enabling the invention to provide for early detection of an intention to leave the bed before there is any significant movement of the occupant's centre of gravity.

The width of the elongated first electrode is ideally less than 10% of the total width of the bed or mattress.

In a preferred embodiment, multiple first electrodes or a segmented first electrode is provided at or close to a longitudinal edge of the bed or mattress, said electrode or electrodes being configured to create several zones of sensitivity for detection of changes in dielectric shift. Preferably, the zones of sensitivity are provided by a series of elongated, ideally parallel, adjacent electrodes of different lengths. More preferably, the series of electrodes progressively increase in length so that each electrode in the series has a zone of sensitivity that is a subset of the adjacent electrode in the series. However, it is to be appreciated that multiple zones of sensitivity may be realised by alternative means.

In one embodiment; the first electrode or electrodes is or are bonded to a self-adhesive tape for attachment of the electrode to the bed or mattress. For example, the self-adhesive tape may be spooled onto a roll or reel for ease of storage and application. Preferably, the sensor is provided with means for its connection to a second electrode and/or a detector.

Alternatively, the first electrode may be woven into the bed mattress or bed linen. It is to be appreciated that the electrode should be positioned within the mattress or bed linen such that it stretches along at least one of the sides of the bed in use.

Preferably, the first electrode comprises or includes antibacterial components and/or is sterilised prior to use.

In an alternative embodiment, the electrode is attached to a chair or to the cover for a chair. In this embodiment, the electrode is preferably positioned substantially centrally on the seat part of the chair, preferably running at least from the middle of the seat part way up the back of the chair.

Preferably, the device includes a remote analyser and controller circuit in communication with the electrodes for detecting the dielectric shift. More preferably, the second electrode is provided within the controller circuit. In the embodiment of the invention having multiple first electrodes, each electrode is preferably provided with its own analyser circuit, with the controller circuit and second (remote electrode) being common to all the first electrodes.

An oscillator may be provided within the controller that senses the dielectric shift that alters the capacitance, the sensor acting as a variable capacitor that alters the resonant frequency. However, it is to be appreciated that alternative means of measuring capacitance are possible and known in the art that would be equally effective, such as an integrated circuit that is designed specifically for measurement of capacitance. The controller may include an input device to enable detection thresholds to be set up. Due to the high sensitivity of the sensor formed by the electrodes, their output is preferably digitised by the analyser/controller with algorithmic techniques being applied to recover useful signal and reject signal drift.

Suitable means should be provided to allow communication between the first electrode and the detector. For example, a cable assembly terminating in an electrical connector may be provided for connecting the controller circuit to the first electrode.

Preferably, the remote analyser, controller circuit and/or second electrode are provided within a housing adapted for attachment to a part of the object, such as the bed, mattress or chair.

The detector of the monitoring device is preferably calibrated to disregard background drift and noise but activate an alarm status upon detection of a pattern of a change in dielectric shift indicative of a person intending to dismount the bed or chair.

A second aspect of the present invention provides a method for monitoring the movement of a load on an object, the method comprising:

attaching a first electrode to an uppermost face of the object substantially parallel to its longitudinal axis;

connecting the first electrode to a remote analyser and controller circuit positioned remotely from said first electrode, the remote analyser and controller circuit including a second electrode for forming a capacitor with the first electrode;

detecting changes in dielectric shift induced in the capacitor by movement of a load on the object; and

activating an alarm status upon detecting a change in dielectric shift induced by movement of the load on the object.

Preferably, the load is a person and the object is a bed, mattress or chair. It is to be appreciated that the electrode may be attached directly to the object or to a cover encasing said object.

Preferably, an alarm status is activated upon detecting a predetermined pattern of dielectric shift indicative of a person intending to dismount the object, such as the bed/mattress or chair.

In a preferred embodiment, the first electrode is attached substantially adjacent a lateral edge of a bed or mattress, i.e. along a long side of the bed. Generally, the method will include attaching a pair of first electrodes to the bed or mattress, the first electrodes being attached to opposing sides of the bed or mattress. The first electrodes may comprise multiple or segmented electrodes to create several zones of sensitivity for detection of changes in dielectric shift. For example, the method may include attaching a series of parallel adjacent first electrodes of different lengths to the object to create several zones of sensitivity for changes in dielectric shift.

The change in capacitance signal or oscillator frequency brought about by the dielectric shift that is induced in the capacitor by movement of the occupant of the bed can be analysed to provide a pattern indicative of a person intending to dismount the bed. It has been found that a pattern in change of capacitance signal or oscillator frequency comprising a sharp increase followed by a more gradual increase, followed by a reduction is indicative of an intention of the occupant to leave the bed. Accordingly, preferably detection of this pattern activates an alarm status. Preferably, patterns are detected for different zones along the longitudinal axis of the object.

Different types of alarm status may be activated by the method of the present invention. For example, detecting a different dielectric shift pattern, such as a sudden increase in oscillator frequency that may indicate that the occupant has rolled from a bed may activate a different visual or audible alarm.

Alternatively, the first electrode may be attached lengthwise along at least part of a seat and/or back of a chair. Preferably, the electrode is attached substantially centrally within the chair. The change in oscillator frequency brought about by the dielectric shift indicative of a person intending to dismount a chair can be analysed to provide a pattern indicative of a person intending to dismount the chair whereby the alarm status is activated upon detection of said pattern.

The method may also include measuring the capacitance at point of installation of the electrodes to provide a baseline against which to monitor the dielectric shift. This enables the method and the device to be tolerant to installation in a wide range of dynamic electrical environments. The method may further include measuring the capacitance of the electrodes to provide a drifting baseline against which to monitor the dielectric shift thereby enabling rejection of low frequency noise and drift, i.e. detecting changes that happen over relatively short timescales while rejecting slow drift that occurs over many minutes or hours.

### BRIEF DESCRIPTION OF THE DRAWINGS

Embodiments of the invention shall now be described, by way of example only, with reference to the accompanying drawings in which:

FIGS. 1A and 1B illustrate a bed fitted with a monitoring device according to one embodiment of the present invention;

FIG. 2 is a perspective view of an antenna for use in a monitoring device according to an embodiment of the present invention;

FIG. 3 illustrates the installation of the antenna shown in FIG. 2 onto a bed;

FIGS. 4A to 4D illustrate the steps generally taken by a person prior to dismounting a bed;

FIG. 5 is a plot of oscillator frequency v time illustrating the signals generated by the steps taken in FIGS. 4A to 4D;

FIG. 6 is a schematic of one embodiment of a circuit diagram for use with the monitoring device of the present invention;

FIG. 7 is a schematic top view of a bed fitted with an antenna for a monitoring device according to another embodiment of the present invention;

FIGS. 8A to 8D illustrate how different load profiles on the bed will intersect different zones of the antenna shown in FIG. 7; and

FIG. 9 is a plot of oscillator frequency v time illustrating the capacitance signals generated by the load profile shown in FIG. 8D for the different zones B to D provided by the antenna of FIG. 7.

### DETAILED DESCRIPTION

The present invention relates to assistive technology for the prevention of inpatient falls. In particular, the invention provides a method and device for monitoring when a patient has an intention to leave a bed or chair rather than notifying healthcare staff that the occupant has already left the bed/chair and that the bed/chair is vacant. This will improve response times and enable preventative actions to be put in place in a timely fashion to reduce the risk of injury occurring to the patient upon leaving the bed or chair.

In a preferred embodiment, the invention is able to monitor an intention to leave the bed by detecting placement of a limb of the occupant near to the perimeter of the mattress. This movement is a preliminary indicator that the occupant may be intending to leave the bed and thus monitoring this will provide an earlier warning than prior art devices and enable preventative measures to be put in place sooner before the occupant actually vacates the bed, being the point when injury is most likely to occur.

The invention relies on a sensor 4 being placed along the side edges of the mattress 2, as illustrated in FIGS. 1A and 1B of the accompanying drawings. The positioning of the sensor at the edge of the mattress is the optimal position to detect movement characteristic of an intent to dismount the bed whilst being largely insensitive to other types of movement by the occupant of the bed. The system comprises of a controller and analyser 6 and an antenna 4. The controller is mounted at the foot or head of the bed 1 and the antenna 4 is unobtrusively attached to the mattress edge in a way that will be undetectable to the occupant. The antenna 4 forms one electrode of a capacitor, the other electrode being within the controller. The arrangement means that there is a detectable shift in the capacitance between these electrodes whenever the occupant places a limb or other body part in proximity to the antenna. This can be sensed by the controller, which evaluates the signal and determines whether an alarm response is appropriate. The system is selective and will not be triggered by the movement of a pillow or bedclothes. The system may be based on a microcontroller, which makes it highly adaptable. For example, the sensitivity can be adjusted easily by the user to minimise false alarms. The sensor could integrate with existing alarm systems or it could host a network-based alarm using wired or wireless network infrastructure.

As shown in FIGS. 1A and 1B, the controller 6 is electrically connected to the antenna 4 by means of a cable assembly 8 with an electrical connector that mates with a socket in the controller. The antenna 4 is formed of an electrically conductive wire, tape or fibre, such as a single- or multi-core electrical wire, braided conductor, metal tape, conductive ink or the like, so that there is a capacitance between it and the electrical ground of the controller electrical circuit. The controller consists of an electrical circuit (described in more detail hereafter in relation to FIG. 6) and a logical device (such as a microprocessor or FPGA) both encapsulated within a common housing that preferably also provides a means to affix the controller/analyser temporarily to the bed.

The antenna 4 is positioned as shown to enable early detection of an intention of the occupant to leave the bed. If the occupant wishes to dismount the bed he must first extend a limb so that it bisects the antenna. The presence of the limb between the antenna 4 and controller 6 will induce a dielectric shift that alters the capacitance. This is sensed in the controller by means of an LC oscillator wherein the sensor acts as a variable capacitor that alters the resonant frequency. The figure shows only a single antenna for reasons of visual clarity, but it is to be appreciated that, ideally, there should be two antennae, one on each side of the mattress. Both antennae could be operated from a single common controller or each antenna could have its own independent controller.

FIGS. 1A and 1B show just one possible embodiment of the sensor. A major advantage of the present invention is that there are no strict constraints on the position or geometry of the sensitive element. This gives the user flexibility to place the various elements of the sensor system in various places in and around the bed to suit the circumstances and to best meet the user's particular needs.

FIG. 2 of the accompanying drawings illustrates a preferred embodiment for an antenna for use in the device of the present invention. The antenna 4 comprises a conductive wire, tape or fibre 4a bonded to a self-adhesive tape 4b. This tape is supplied wound onto a reel or roll for ease-of-use. At the free end of the roll of tape there is an electrical cable assembly 4c terminated in an electrical plug that allows the antenna to be connected to the controller. This embodiment is suited to being a disposable item. The major advantages of this embodiment are its convenience, ease of installation, flexibility and adaptability, its intrinsic hygiene and very low cost of manufacture.

FIG. 3 illustrates the antenna of FIG. 2 being installed in a bed 1. The free end of the tape is bonded to the foot of the bed, in close proximity to the controller 6 and the cable connector is inserted into the socket therein. The self-adhesive tape 4b is then un-rolled from its reel while simultaneously being bonded to the mattress. Preferably, the roll of tape would be supplied sized to suit the length of the bed but the antenna may also be cut to length as appropriate. This illustrates a major benefit of the present invention in that there are no particular constraints on the position or geometry of the sensor element.

It is advantageous to position the sensitive element longitudinally at the edge of a mattress because, in this position, it is sensitive to actions that are characteristic of an intent to leave the bed but significantly less sensitive to normal patient movement. The present invention facilitates this advantageous positioning because its sensitive element, being a single wire, tape or filament, is extremely narrow and can occupy a very narrow swathe of the mattress, preferably being less than 10% of the total mattress width. By contrast, existing sensors occupy a much wider swathe, typically being positioned across the entire width of the mattress and occupying 10 to 20% of total mattress area. This makes them less able to discriminate between normal movement of the occupant and an actual intent to leave the bed when positioned as described.

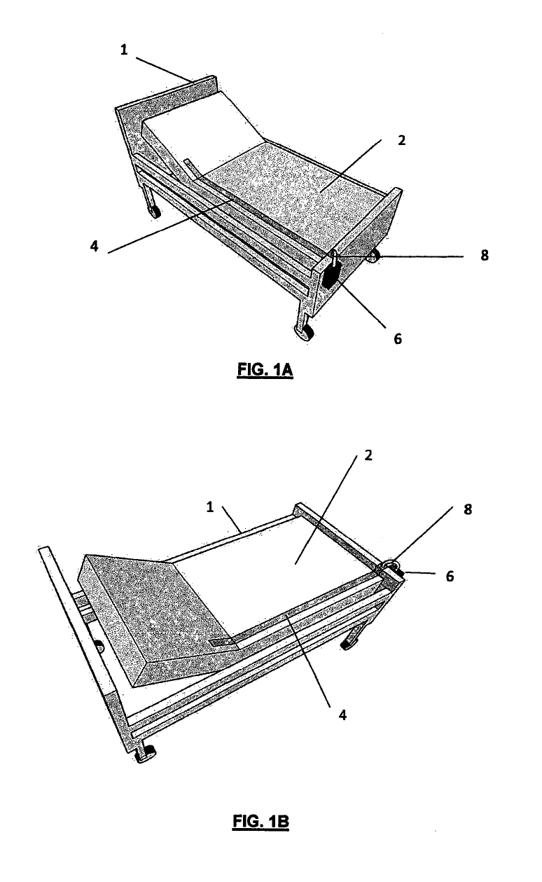

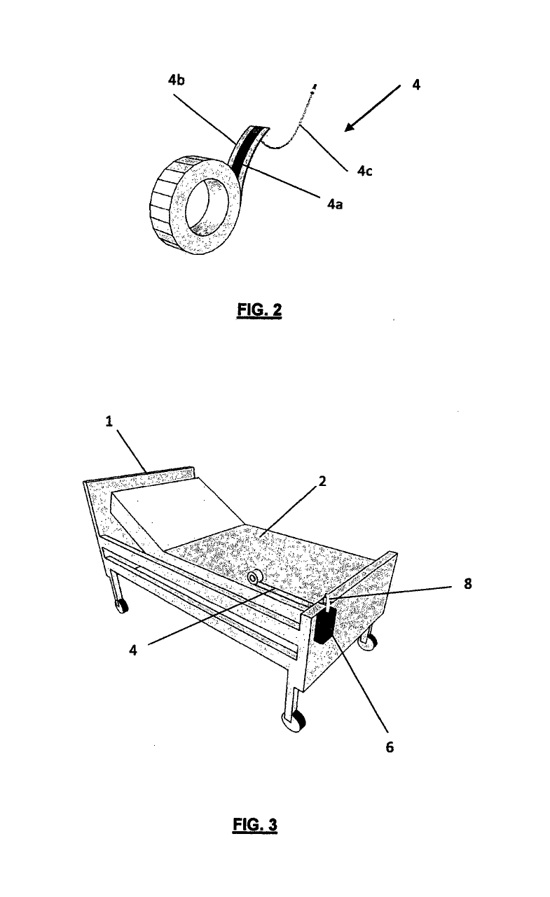

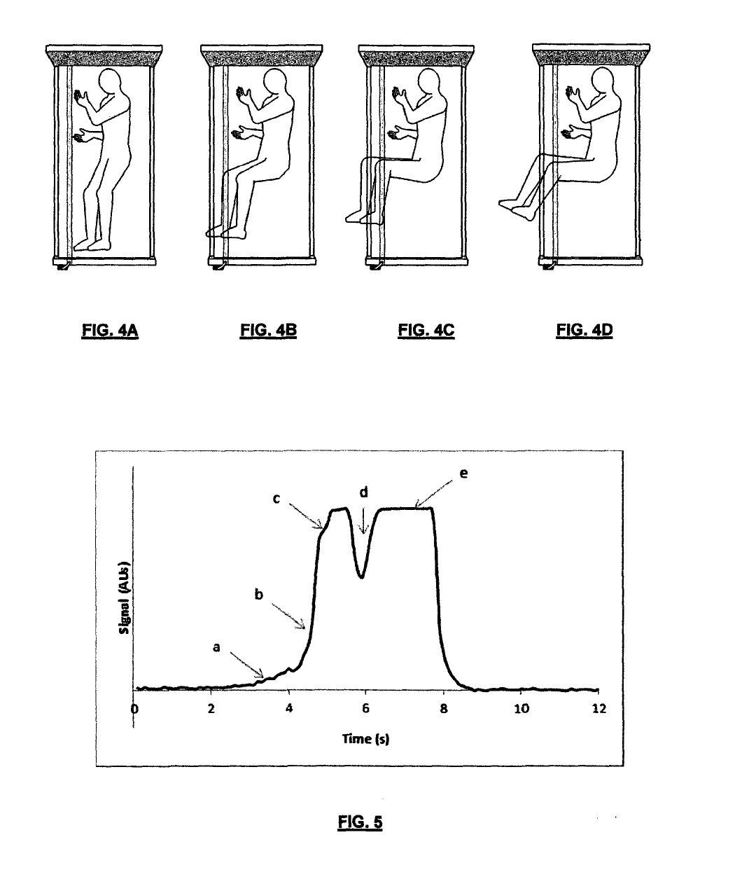

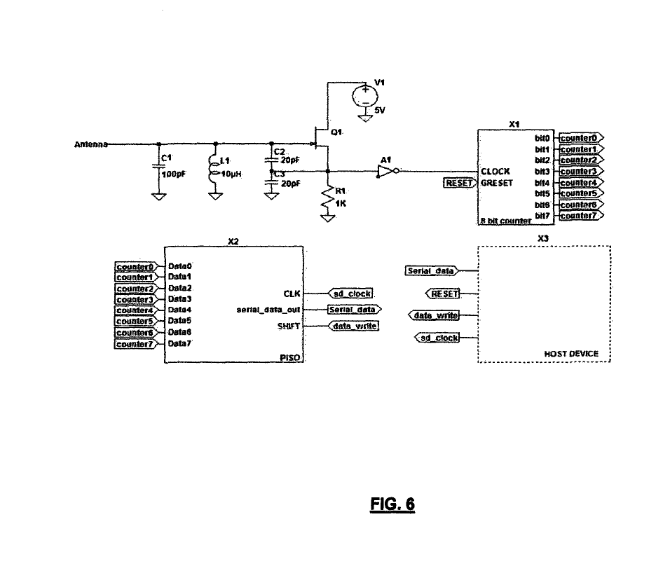

FIGS. 4A to 4D show, solely by way of example, how the advantageous characteristics of the present invention may be exploited to maximise probability of detection and prevent false alarms. It is based on a model of movement that is typical of less mobile persons attempting to dismount a bed in which both legs are extended beyond the edge of the mattress and used as a counterweight to aid the raising of the upper body into an upright position. FIG. 5 illustrates the variation in oscillator frequency over time during these movements.

First, there is an increase in the oscillator frequency as the occupant makes his or her initial movement to extend a leg toward the mattress edge (see FIG. 4a, represented by region “a” on FIG. 5). This is detectable above the noise floor of the sensor but it is not distinguishable from normal motion of the patient within the bed.

As the lower leg bisects the antenna, there is a sudden and very rapid increase in the oscillator frequency (FIG. 4b and rise “b” in FIG. 5). The signal is large at this instant because the lower leg intersects with a significant length of the antenna. For the present invention, this signal would be considered abnormal because it indicates that the patient has placed a part of his or her body at the mattress edge;

even if this does not signify intent to leave the bed, it would indicate assistance is required since the occupant might suffer discomfort if left unaided. Existing sensors occupy a much larger swathe of the bed so that this kind of signal could easily result from normal movement of the patient where he or she places a limb near (rather than adjacent to or over the edge of) the mattress.

In case of a genuine intent to leave the bed, the patient's next movement will typically be to further flex the hips and raise the lower leg and knees up along the mattress edge towards the head of the bed (as shown in FIG. 4c). This leads to a further increase in the oscillator frequency, see “c” in FIG. 5. In this case the increase is relatively slow because the area of intersection between the limb and the antenna is fixed and the capacitance is modulated only by the changing position of the limb. It is not possible to detect motion 4c with existing capacitive sensors where there is a multiplicity of adjacent electrodes. Such sensors are sensitive only to the area of intersection between the limb and the element and not to the position within the bed at which the intersection occurs.

Next the occupant will tend to extend the lower leg beyond the edge of the mattress (FIG. 4d) in preparation that the legs might be swung down to provide a counterweight that will help him or her raise the upper body to achieve an upright sitting position. Once the lower leg is extended in the way described, the intersection between the antenna and the limb is reduced, which leads to a reduction in the oscillator frequency, see “d” in FIG. 5.

Finally, the occupant will typically rise into an upright sitting position (not shown). At this point, both upper legs are in contact with the antenna and, since it is common for the legs to be positioned at an oblique angle to the mattress (rather than normal to the mattress edge) there is a large intersection between the legs and the antenna. Hence, the oscillator frequency increases again (rise “e” in FIG. 5).

The sequence of signals described, b to d, in FIG. 5 is characteristic of a person having intent to leave the bed. Signal e confirms that the occupant is in an upright attitude and may dismount the bed imminently. The data in FIG. 5 demonstrates that the present invention is capable of identifying the sequence of events described. Detection could be automatic by software algorithms or by an experienced human observer viewing the signal data. In the latter case, the observer may be remote from the bed and viewing the signal data by telemetry transmitted by the controller/analyser.

It is to be appreciated that detection of alternative patterns may activate an alarm status. For example, rolling of an individual from a bed would provide a different dielectric shift pattern but would still need to raise an alarm status. A sudden increase in oscillator frequency, as the mass of the whole body passes over the length of the antenna, could also result in activation of an alarm. The alarm could be different, for example, of a different tone or colour, to indicate that more than one carer is required to attend to administer all necessary post-fall assessments or treatments and assist in placing the individual back in bed.

FIG. 6 of the accompanying drawings provides one example of how the dielectric shift between the electrodes of the capacitor may be monitored to provide a sensitive and selective system. The figure shows an electrical schematic of one possible embodiment of the controller/analyser circuit that illustrates the detection principle and the electrical function of the antenna. The capacitors C1, C2 and C3 and inductor L1 form the tank circuit of a Colpitts LC oscillator. The FET transistor Q1 and its load resistor R1 form source-follower with C2 and C3 providing a feedback network to sustain the oscillations. The example circuit oscillates at approximately 4.8 MHz, which has been found to work well in practice, but the frequency can be readily tuned by adjustment of values of C1 or L1. The resonant frequency should be chosen to minimise the radiated susceptibility and any effects of radiated emissions.

The stray capacitance between the antenna and the circuit electrical ground in the controller acts to modify the effective capacitance within the tank circuit. This changes the resonant frequency of the oscillator. The antenna is thereby acting as a variable capacitor that tunes the oscillator frequency.

Inverting buffer amplifier Al converts the near-sinusoidal oscillations at its input into a square wave at its output, which is then used as the clock for a digital counter X1. This results in the counter incrementing once for each period of the oscillator. The parallel data from the counter is sampled and then serialised by parallel-in-serial-out register X2 that converts the count into a serial bitstream for transmission to a host device X3, such as a microprocessor.

The counter X1 is first reset by asserting the RESET signal and then sampled a fixed time later by application of a signal pulse on data_write. The sampled counter value is proportional to the frequency of the oscillator and therefore contains information about the stray capacitance of the antenna. This data may be used to detect the proximity of the bed occupant to the antenna. The data can be clocked out of the serialiser X2 on the data port (serial_data) using an asynchronous clock (sd_clock). Preferably, these functions would be performed by the host device X3 such as a computer, FPGA or microprocessor. Such a host could readily provide the digital discrete timing signals that define the interval (RESET, data_write) using GPIOs, for example. Importantly, the host device offers a platform to run software algorithmically, separating useful signals from drift and noise.

The counter X1 and serialiser X2 are shown separately from the host X3 in order to illustrate the principle of operation of the example circuit. However, many microprocessors incorporate integral digital counters and timers that could perform all of the digital functions of the circuit, and indeed such an arrangement is preferred.

The sensitivity of the example circuit depends on the time interval between resetting and sampling the counter X1. There will be no change in the digital output until there is at least one half-cycle difference in the number of oscillations within the interval. Therefore, a longer interval results in a greater change in the digital output for a given change in capacitance. If the counter of the example circuit is sampled 300 microseconds after reset, then each Least Significant Bit (LSB) is equivalent to a change of approximately 45 fF at the sensor. The sensitivity can be increased by simply increasing or decreasing the interval. It is important that the counter X1 recirculates to zero on overflow; otherwise the interval is limited to a maximum duration equivalent to 255 resonator oscillation periods.

The maximum full-scale range of the example circuit is approximately 11.5 pF (256 possible values at 45 fF per LSB) and the practical dynamic range is likely to be significantly less. If required, the counter X1 can be cascaded with a second 8-bit counter to give a 16-bit resolution and so forth until the desired dynamic range is achieved.

In this manner, the variation in oscillator frequency can be monitored by the host device. The device may be calibrated to disregard background noise and drift but to activate an alarm, be it audible and/or visual, when a particular pattern in the variation of oscillator frequency is detected that is indicative of a person's intention to dismount a bed. Ideally, the capacitance between the electrodes is measured at the point of installation and all subsequent measurements are made relative to this static baseline. The capacitance is measured periodically at predetermined intervals of time and all measurements are made relative to the slowly drifting baseline to enable low frequency noise and drift to be rejected, for example with changes detected over short time periods being accepted while those that occur over longer periods of time being rejected. This enables the device of the present invention to more accurately detect an intention of a person to dismount a bed before they actually do so and minimises false alarms brought about by non-significant movement of the person within the bed.

While FIG. 6 describes the use of a LC oscillator and counter as the capacitance measuring circuit, it is to be appreciated that alternative means may be utilised to measure changes in capacitance. For example, commercially available capacitive sense integrated circuits may be used.

An alternative embodiment of the present invention detects an intention of a person to leave a chair. The antenna or electrode is run at least partway along the back and/or seat of the chair and connected to a controller/analyser containing a second electrode as hereinbefore described. A sequence of signals characteristic of a person having intent to leave the chair are determined, for example as generated when a person moves their bottom forwards, then torso, before raising their bottom from the chair, wherein detection of the aforesaid signals activates an alarm. Again, detection could be automatic by software algorithms or by an experienced human observer viewing the signal data.

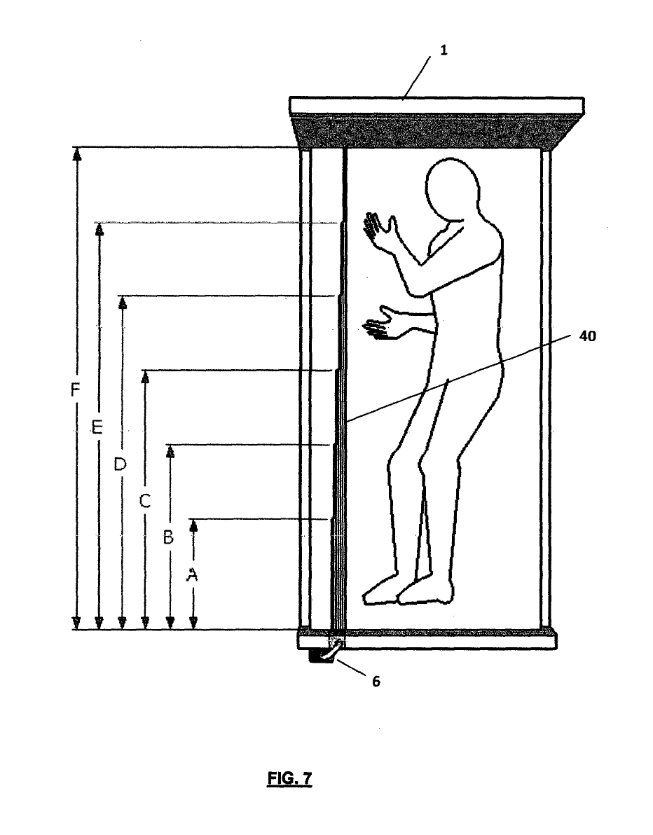

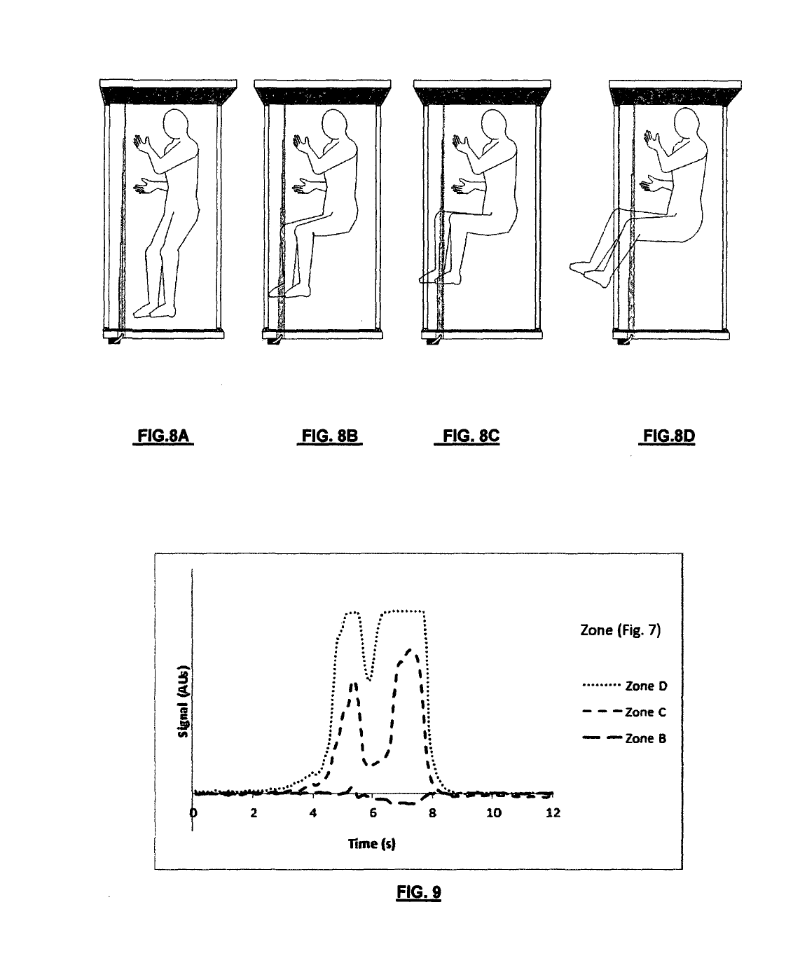

FIGS. 7 to 9 of the accompanying drawings illustrate another embodiment of the present invention. Identical features to those shown in the previous embodiment of FIGS. 1 to 3 are given the same reference numerals and only the differences will be discussed in detail. The antenna 40 is again attached to a longitudinal edge of a bed 1 but is segmented into several longitudinal zones of sensitivity by the provision of multiple duplicate antennae placed in parallel, each one being of a different length A, B, C, D, E and F (see FIG. 7). This increases the spatial information contained in the signal so that, for example, an arm intersecting the antenna may be distinguished from a leg by its position in the bed provided by reference to the detection of oscillator patterns in different zones provided by the antenna 40.

This is illustrated in FIG. 8D. Where a limb intersects the sensor, it will register a signal in some of the antennae while others will not be intersected due to their shorter length. In the position of the individual shown in FIG. 8D, there will be a strong signal from antennae C to F, a very weak signal from the antenna B and no signal from antenna A. This locates the position of the intersection to within zones B-C and is indicative of movement prior to dismount of an individual from the bed. FIGS. 8A to 8C illustrate how other positions would alter the signals generated by the different antenna zones A to F.

FIG. 9 shows real measured data from the sensor shown in FIG. 7 in the same manner as that shown for the antenna of FIGS. 1 to 3 in FIG. 5 whereby the postural profile of the load (patient) on the bed can be deduced from the presence and intensity of the signals detected for each of the zones A to F (note in FIG. 9 the signals for zones 40A, 40E and 40F are omitted for visual clarity). Thus, for the postural profile of FIG. 8D, the intensity of the signal present in zone C and absence of the same signal in zone B shows that the interaction has occurred in zone C. The greater intensity of the signal in zone D than zone C also shows that there has been an interaction in zone D. This signal pattern identifies that there has been interaction between the load and the sensor in a lower portion of the centre of the bed (zone C-D), making it far more likely that the lower limbs have been moved rather than the arms providing a higher likelihood of the patient dismounting the bed. Additional details of the profile of the patient can be deduced from the intensity of the signals as shown in FIG. 9.

The embodiment of FIGS. 7 to 9 increases the spatial information contained within a signal. This additional discrimination should further reduce the occurrence rate of false alarms since it is able to differentiate between signals generated by movement of different parts of the body, such as an arm or leg.

While the invention has been discussed mainly in relation to hospital usage, it is clear that the monitoring device could be used outside of the hospital environment, such as in nursing homes and within the domestic setting.

The monitoring device of the present invention provides a sensor as one electrode of a capacitor with the second electrode being positioned remotely from the sensor (or first electrode(s)). The dielectric shift is detected by means of the sensor's capacitance to this remote second electrode. This differs to earlier devices in which the sensor is realised by a pair of electrodes and the dielectric shift is sensed by means of their mutual capacitance. This present arrangement allows for a filamental (i.e. wire like) electrode for attachment to the bed or other object which allows easier optimal positioning of the electrode adjacent an edge of the bed and enables a narrower swathe of the bed to be occupied by the sensor, reducing unintentional alarms that may occur as a result of movement of the occupant of within bed.

1.-26. (canceled)

27. An occupancy monitoring device for detecting movement of a load on a bed or mattress, the device comprising a sensor in the form of a capacitor having a first electrode adapted for attachment to at least part of the bed, mattress or a covering therefor and a second electrode adapted for positioning remotely from the first electrode, the device further comprising a detector for detecting dielectric shift induced in the capacitor by movement of a load on the bed or mattress, wherein the first electrode comprises multiple zones of sensitivity for detection of changes in dielectric shift, the multiple zones being provided by a series of elongated, parallel first electrodes of different length, wherein each length of electrode represents a different zone of sensitivity.

28. A monitoring device as claimed in claim 27 wherein the or each first electrode comprises a single conductor elongated wire, film, tape or fibre.

29. A monitoring device as claimed in claim 27, wherein the total width of the elongated first electrode or electrodes is less than 10% of the total width of the bed or mattress.

30. A monitoring device as claimed in claim 27 wherein the first electrode is bonded to a self-adhesive tape for attachment of the electrode to the bed, mattress of covering therefor.

31. A monitoring device as claimed in claim 27 wherein the first electrode is woven into a bed mattress or bed linen.

32. A monitoring device as claimed in claim 27 further comprising a remote analyser and controller circuit in communication with the electrodes for detecting the dielectric shift.

33. A monitoring device as claimed in claim 32 wherein the second electrode is provided within the controller circuit.

34. A monitoring device as claimed in claim 32 further comprising a cable assembly terminating in an electrical connector for connecting the controller circuit to the first electrode.

35. A monitoring device as claimed in claim 32 wherein the remote analyser, controller circuit and second electrode are provided within a housing adapted for attachment to a part of the bed or mattress to which the first electrode is attached, remote from said first electrode.

36. A monitoring device as claimed in claim 27 wherein the detector is calibrated to disregard background drift and noise but activates an alarm status upon detection of a pattern of a change in dielectric shift indicative of movement of a person intending to dismount the bed or mattress.

37. A method for monitoring the movement of a load on a bed or mattress the method comprising:
attaching a series of elongated, parallel first electrodes of different lengths to an uppermost face of the bed or mattress of a covering therefor substantially parallel to its longitudinal axis to create multiple zones of sensitivity, each electrode of a different length representing a different zone of sensitivity;
connecting the first electrode to a remote analyser and controller circuit positioned remotely from said first electrode, the remote analyser and controller circuit including a second electrode to form a capacitor;
detecting changes in dielectric shift induced in the capacitor by movement of the load on the bed or mattress; and
activating an alarm status upon detecting a change in dielectric shift induced by movement of the load on the bed or mattress.

38. A method according to claim 37 further comprising attaching the first electrode substantially adjacent a lateral edge of the bed or mattress.

39. A method according to claim 37 wherein the alarm status is activated upon detecting a predetermined pattern of dielectric shift indicative of a person intending to dismount the bed/mattress.

40. A method according to claim 37 wherein the alarm status is activated upon detecting a pattern in a change of capacitance caused by the dielectric shift comprising a sharp increase in capacitance signal followed by a more gradual increase in the signal, followed by a reduction in the signal.

41. A method according to claim 40 wherein an alternative alarm status is activated upon detecting a sharp increase in capacitance signal indicative of a person rolling or falling from the bed/mattress.

42. A method according to claim 37 further comprising measuring the capacitance at point of installation of the electrodes to provide a baseline against which to monitor the dielectric shift.

43. A method according to claim 37 further comprising periodically measuring the capacitance of the electrodes to provide a drifting baseline against which to monitor the dielectric shift thereby enabling rejection of low frequency noise and drift.
